(19) United States
(12) Patent Application Publication
(10) Pub. No.: US 2019/0076125 A1
(43) Pub. Date: Mar. 14, 2019

(54) APPARATUSES, METHODS, AND SYSTEMS FOR ANNOTATION OF MEDICAL IMAGES

(71) Applicant: KONINKLIJKE PHILIPS N.V., Eindhoven (NL)

(72) Inventor: STEFAN BURTON ROGER, EINDHOVEN (NL)

(21) Appl. No.: 15/765,610

(22) PCT Filed: Sep. 26, 2016
(86) PCT No.: PCT/IB2016/055727

§ 371 Date: Apr. 3, 2018

Related U.S. Application Data
(60) Provisional application No. 62/238,758, filed on Oct. 8, 2015.

(51) Int. Cl. A61B 8/00; G16H 30/20

(52) U.S. Cl. CPC A61B 8/468; G06F 19/00; G16H 30/20

(57) ABSTRACT

A method and system for annotating images is disclosed. A user may annotate images with a touch screen of a medical imaging system. The annotations may be free-form drawings by the user. The annotations may be displayed with the image on the touch screen and another display of the medical imaging system. The annotations may be associated with metadata that may provide location, diagnostic, and/or other information. The metadata, annotations, and image may be stored in a memory of the medical imaging system. The image may be retrieved from the memory for later review based on the metadata.

### BACKGROUND

During a clinical examination, users of a medical imaging system often wish to add notations to images. Users may add annotations to live images displayed on a screen and/or add annotations to already-acquired images that are being reviewed. The purpose of these annotations may include giving a title or description to individual images, highlighting a particular area of the image, and indicating what part of anatomy is being imaged. For real-time imaging modalities, such as ultrasound imaging, speed and efficiency of imaging as well as annotating images are important. Given the variety of patients imaged by medical imaging systems, the annotation methods also need to be flexible to catch peculiar or unexpected anomalies in patients. For example, during a surgery, an ultrasound technician may wish to highlight a pocket of fluid to be viewed by a surgeon.

Existing methods for entering annotations of medical images include: typing with the keyboard, pressing and/or rotating knobs on a control panel, pressing buttons on a touch screen and/or control panel, and/or moving the trackball. Several actions and potentially many movements using input devices are required for a user to complete their desired annotation, reducing efficiency.

Free text annotations can be added to the image using a keyboard, as in a normal text-processing application. However, the size and styling of the text is limited to what the imaging system specifies based on manufacturer input or limited customized input. The manufacturer may limit text annotations to a fixed set of options. The user may not be able to enter all the characters and/or symbols they desire due to keyboard and character set limitations.

Graphic-based annotations available on imaging systems today are often limited to a fixed selection of annotations to choose from while conducting an exam. Moreover, users cannot add arbitrary or custom graphics to the image with the imaging system.

In clinical practice, inefficiency in annotating images may be exacerbated when imaging systems partition all types of annotations into different system modes, requiring extra steps when adding multiple types of annotations. For example, marking some pathology with an arrow and a text label may be a five-step process: Activating “arrow annotation” mode, positioning the arrow, activating “text label” mode, positioning the text cursor, and typing the text annotation. This workflow may be especially tedious when the user wants to change the annotations often or use many different annotations during one patient exam.

### SUMMARY

An example medical imaging system according to an embodiment of the disclosure may include a touch screen that may be configured to receive annotation input from a user, wherein the annotation input may be associated with an image acquired by the medical imaging system, a graphics processor that may be configured to receive the annotation input from the touch screen and may generate a graphic overlay based, at least in part, on the annotation input, wherein the graphic overlay may be associated with the image as an annotation, a display that may be configured to display the image and the annotation, and a memory that may be configured to store the image and the annotation.

An example method of annotating an image according to an embodiment of the disclosure may include receiving annotation input from a touch screen, wherein the annotation input is associated with the image, generating a graphic overlay corresponding to the annotation input, wherein the graphic overlay corresponding to the annotation input is an annotation, and providing the graphic overlay to the touch screen.

### BRIEF DESCRIPTION OF THE DRAWINGS

FIG. 1 is a functional block diagram of an ultrasound imaging system according to an embodiment of the disclosure.

FIG. 2 is a schematic illustration of an ultrasound imaging system according to an embodiment of the disclosure.

FIG. 3 is a graphical display of a touch screen according to an embodiment of the disclosure.

FIG. 4 is a graphical display of a display according to an embodiment of the disclosure.

FIG. 5 is a flow chart of a method according to an embodiment of the disclosure.

FIG. 6 is a flow chart of a method according to an embodiment of the disclosure.

### DETAILED DESCRIPTION

The following description of certain exemplary embodiments is merely exemplary in nature and is in no way intended to limit the invention or its applications or uses. In the following detailed description of embodiments of the present systems and methods, reference is made to the accompanying drawings which form a part hereof, and in which are shown by way of illustration specific embodiments in which the described systems and methods may be practiced. These embodiments are described in sufficient detail to enable those skilled in the art to practice the presently disclosed systems and methods, and it is to be understood that other embodiments may be utilized and that structural and logical changes may be made without departing from the spirit and scope of the present system.

The following detailed description is therefore not to be taken in a limiting sense, and the scope of the present system is defined only by the appended claims. The leading digit(s) of the reference numbers in the figures herein typically correspond to the figure number, with the exception that identical components which appear in multiple figures are identified by the same reference numbers. Moreover, for the purpose of clarity, detailed descriptions of certain features will not be discussed when they would be apparent to those with skill in the art so as not to obscure the description of the present system.

According to an exemplary embodiment of the disclosure, a user (e.g., ultrasound technician, clinician) may freely draw annotations on a live or previously acquired image using a touch screen as an input device. A live image may be an image currently being acquired and displayed by a medical imaging device. For example, an ultrasound imaging system may display an image based on echo signals being received by an ultrasound probe. Examples of images may include a 2D image, a plane of a 3D image, and/or a rendering of a 3D volume. In some embodiments, the user may draw annotations on an image using their finger and/or a stylus to interact with the touch screen. The user may then save the annotations as part of the image for later viewing. The annotations may be shown on the touch screen and another display of a medical imaging device. The touch screen and display may display corresponding images and annotations. This may facilitate sharing of information between multiple users. For example, an ultrasound technician may annotate a live image with the touch screen of the medical imaging device by circling a blood vessel. The display of the medical imaging device may be turned toward a clinician performing a medical procedure for easier viewing. The clinician may see the same live image and annotations of the ultrasound technician on the display. The clinician may see the annotations of the live ultrasound image on the display as they are input by the ultrasound technician on the touch screen.

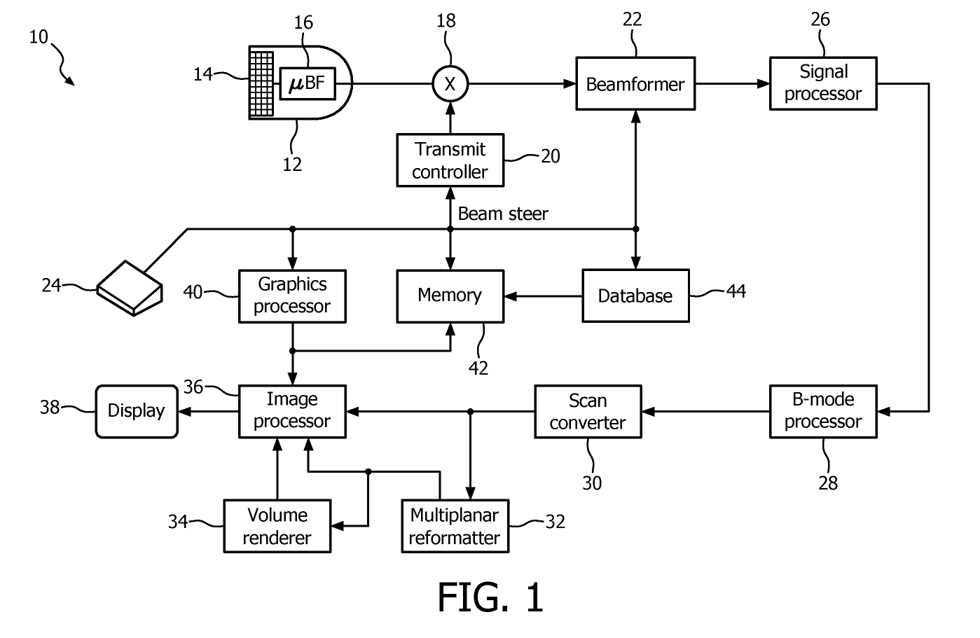

Referring to FIG. 1, an ultrasound imaging system 10 constructed in accordance with the principles of the present disclosure is shown in block diagram form. In the ultrasonic diagnostic imaging system of FIG. 1, an ultrasound probe 12 includes a transducer array 14 for transmitting ultrasonic waves and receiving echo information. A variety of transducer arrays are well known in the art, e.g., linear arrays, convex arrays or phased arrays. The transducer array 14, for example, can include a two dimensional array (as shown) of transducer elements capable of scanning in both elevation and azimuth dimensions for 2D and/or 3D imaging. The transducer array 14 is coupled to a microbeamformer 16 in the probe 12 which controls transmission and reception of signals by the transducer elements in the array. In this example, the microbeamformer is coupled by the probe cable to a transmit/receive (T/R) switch 18, which switches between transmission and reception and protects the main beamformer 22 from high energy transmit signals. In some embodiments, the T/R switch 18 and other elements in the system can be included in the transducer probe rather than in a separate ultrasound system base. The transmission of ultrasonic beams from the transducer array 14 under control of the microbeamformer 16 is directed by the transmit controller 20 coupled to the T/R switch 18 and the beamformer 22, which receives input from the user's operation of the user interface and/or control panel 24. The user interface and/or control panel 24 may include a touch screen in some embodiments. One of the functions controlled by the transmit controller 20 is the direction in which beams are steered. Beams may be steered straight ahead from (orthogonal to) the transducer array, or at different angles for a wider field of view. The partially beamformed signals produced by the microbeamformer 16 are coupled to a main beamformer 22 where partially beamformed signals from individual patches of transducer elements are combined into a fully beamformed signal.

The beamformed signals are coupled to a signal processor 26. The signal processor 26 can process the received echo signals in various ways, such as bandpass filtering, decimation, I and Q component separation, and harmonic signal separation. The signal processor 26 may also perform additional signal enhancement such as speckle reduction, signal compounding, and noise elimination. The processed signals are coupled to a B mode processor 28, which can employ amplitude detection for the imaging of structures in the body. The signals produced by the B mode processor are coupled to a scan converter 30 and a multiplanar reformatter 32. The scan converter 30 arranges the echo signals in the spatial relationship from which they were received in a desired image format. For instance, the scan converter 30 may arrange the echo signal into a two dimensional (2D) sector-shaped format, or a pyramidal three dimensional (3D) image. The multiplanar reformatter 32 can convert echoes which are received from points in a common plane in a volumetric region of the body into an ultrasonic image of that plane, as described in U.S. Pat. No. 6,443,896 (Detmer). A volume renderer 34 converts the echo signals of a 3D data set into a projected 3D image as viewed from a given reference point, e.g., as described in U.S. Pat. No. 6,530,885 (Entrekin et al.) The 2D or 3D images are coupled from the scan converter 30, multiplanar reformatter 32, and volume renderer 34 to an image processor 36 for further enhancement, buffering and temporary storage for display on an image display 38. The graphics processor 40 can generate graphic overlays for display with the ultrasound images. These graphic overlays can contain, e.g., standard identifying information such as patient name, date and time of the image, imaging parameters, and the like. For these purposes the graphics processor receives input from the user interface 24, such as a typed patient name. The user interface 24 can also be coupled to the multiplanar reformatter 32 for selection and control of a display of multiple multiplanar reformatted (MPR) images. In some embodiments, the graphics processor 40 may be implemented as multiple processors.

In accordance with at least some of the embodiments of the disclosure, the graphic overlays generated by the graphics processor 40 may include annotations input by the user via a touch screen of the user interface 24. The graphics processor 40 and/or image processor 36 may store images and/or the graphic overlays to a memory 42. In some embodiments, a user may select images to be stored via the user interface 24. The images and/or graphic overlays stored in the memory 42 may be retrieved for later viewing on the display 38 and/or transferred to another device (e.g., USB drive, personal computer, electronic medical records system, data storage server) for later viewing.

A user may acquire an image with an ultrasound probe 12 and view the acquired image on a display 38 and/or on a touch screen that is included with the user interface 24. The user may make annotations on the image using their finger, stylus, and/or other input device on the touch screen. The user may annotate a live image or annotate an image that has already been saved to memory 42. The annotations may be dynamically integrated with the image by the graphics processor 40 such that the image and annotation may be simultaneously displayed on the display 38 and touch screen. In some embodiments, annotations made via traditional methods (e.g., track ball, keyboard, and/or other input of the user interface 24) may be made on the same image that annotations are made via the touch screen. The image may be saved with the annotations made via all user input types to the memory 42.

In some embodiments, the graphic overlays including the annotations and the acquired image may be saved in the memory 42 as multiple “layers.” The acquired image may be one or more layers, and the graphic overlays may be one or more layers. For example, patient information may be contained in a layer while annotations input through a touch screen may be stored as a separate layer. A user may be able to select which layers to view. For example, a user may view all layers, which may display the acquired image with the annotations and patient information. The user may then elect to inhibit display of the annotation layer to view the acquired image unobstructed by the annotations.

In some embodiments, the annotations input by the user via the touch screen may be associated with metadata. The metadata may be saved with the image in the memory 42 in the same file and/or as a separate file associated with the image file in the memory 42. In some embodiments, the metadata may indicate where in the image the annotation is located. In some embodiments, the metadata may indicate anatomy the annotation is associated with. In some embodiments, the metadata may flag an image as annotated to allow the image to be tracked over time. For example, a researcher may retrieve images from past exams that include annotations from a set of all the images from past exams stored for a patient based on the flag. The researcher may then be able to review only those images with annotations. This may facilitate quick retrieval and review of relevant images for a condition of a patient being monitored over time.

The metadata may be compliant with a standard, for example, the Digital Imaging and Communications in Medicine (DICOM) standard. The DICOM standard includes a meta tag that may enable a specific diagnostic code, either for diagnostic outcomes tracking or medical billing purposes. In some embodiments, the standards and/or other data may be stored in a database 44 for use as metadata to be associated with the annotations. In some embodiments, the graphics processor 40 may automatically link the annotations with the metadata. In some embodiments, a user may select data to be associated with the annotations. For example, the user may input the specific anatomy the annotation is associated with, the feature being annotated (e.g., fluid pocket, tumor, scar tissue), and/or type of annotation (e.g., measurement, note, warning).

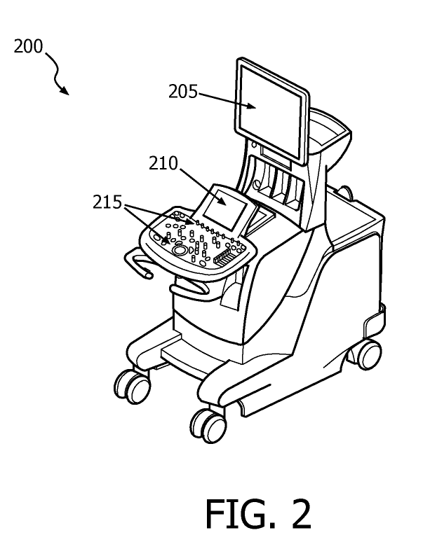

FIG. 2 is a schematic illustration of an ultrasound imaging system 200 according to an embodiment of the disclosure. The ultrasound imaging system 200 may include some or all of the elements of the ultrasound imaging system 10 shown in FIG. 1 in some embodiments. The ultrasound imaging system 200 may include a display 205 that may be positioned by a user. The display 205 may be used to implement display 38 of FIG. 1. In some embodiments, the display 205 may be a flat panel display. The display 205 may be articulated to be viewed over a wide range of viewing positons. An example of an ultrasound system having an articulated flat panel display that may be used to implement one or more embodiments of the disclosure may be found in European Patent EP 1713396. The ultrasound imaging system 200 may further include a touch screen 210 and a control panel 215. The touch screen 210 and/or control panel 215 may be used to implement the user interface 24 of FIG. 1. The touch screen 210 and/or control panel 215 may be used to control image acquisition, imaging parameters, storing data, annotating images, and/or other imaging parameters. The control panel 215 may include one or more control elements for controlling the ultrasound imaging system 200. In the embodiment shown in FIG. 2, the control panel 215 includes a keyboard, a track ball, control knobs, and switches. Other embodiments of the control panel 215 may include more or fewer control elements. Other embodiments of the control panel 215 may include different elements than those shown in FIG. 2. For example, the control panel 215 may include a track pad, one or more rocker switches, and/or microphone. In some embodiments, the control panel 215 may be used to control what is displayed on the display 205 and/or touch screen 210. In some embodiments, the touch screen 210 may be used to control what is displayed on the touch screen 210 and/or display 205.

A user may acquire an image with an ultrasound probe and view the acquired image on a display and/or on a touch screen. To add an annotation to the image, the user may select an option on the touch screen to enter an annotation mode. The user may then make annotations on the image using their finger, stylus, and/or other input device on the touch screen. The user may annotate a live image or annotate an image that has already been saved to memory. The annotations may be dynamically integrated with the image by a graphics processor such that the image and annotation may be simultaneously displayed on multiple displays (e.g., both touch screen 210 and display 205 of imaging system 200). The user may save the acquired image and annotations to a memory and/or export to another location (e.g., e-mail, network drive). In some embodiments, annotations made via traditional methods (e.g., icons, text, and/or symbols selected via the track ball, keyboard, and/or other user interface) may be made on the same image that annotations are made via the touch screen. The image may be saved with the annotations made via all user input types.

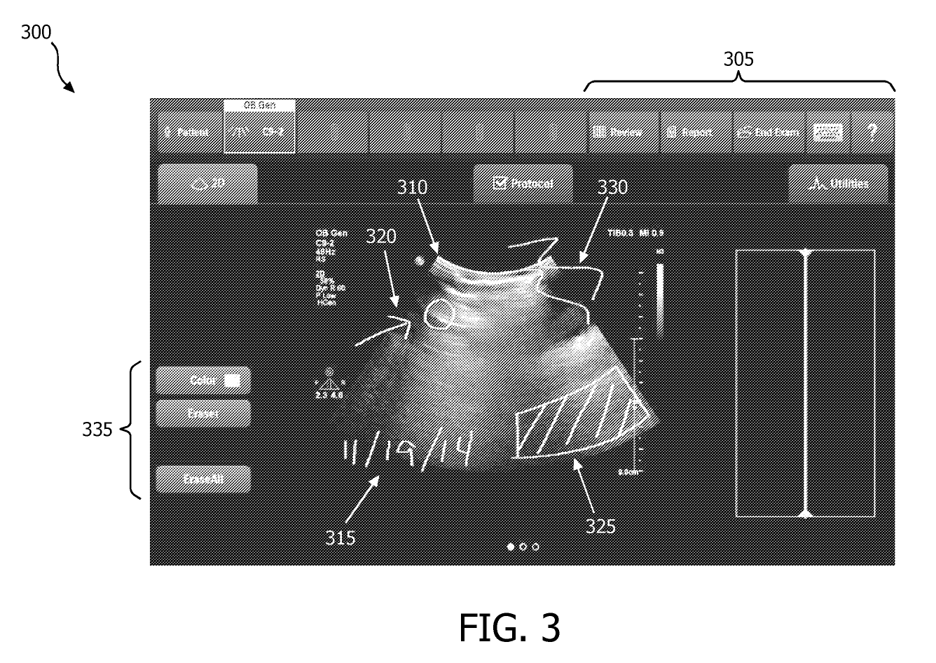

FIG. 3 is an example graphical display 300 of a touch screen of a medical imaging system according to an embodiment of the disclosure. The graphical display 300 may include one or more buttons 305 that may be selected by a user to control a medical imaging system such as medical imaging system 10 and/or 200. For example, the buttons may be used to open help menus, save an image, open previously saved images, and/or other functions. The graphical display 300 may further include an image 310. The image may be a live image or a previously saved image acquired by a probe of the medical imaging system. The user may annotate the image with the touch screen. The annotations may be freeform drawings in some embodiments. The graphical display 300 of FIG. 3 shows several example annotations 315, 320, 325, 330 input by a user via the touch screen. The example annotations include a date 315, a circle and arrow 320, a shaded area 325, and an outline of a feature 330. The annotations shown in FIG. 3 are for exemplary purposes only, and the annotations the user may input are not limited to those shown in FIG. 3. The graphical display 300 may include buttons 335 for controlling the annotations input by the user. For example, the user may be able to erase previously made annotations and/or select the color of an annotation. Other options may also be provided (e.g., line thickness, symbols, copy/paste).

In some embodiments, the user may be able to use gestures on the touch screen to make and/or adjust annotations. For example, a user may be able to double tap a shape drawn by the user to automatically fill in the shape with shading. In another example, a user may drag two fingers across the touch screen in order to expand or contract an annotation.

In some embodiments, the user may associate an annotation with a specific anatomy. The annotation may be propagated across the image and/or images such that the annotation appears in all views that include the specific anatomy. In some embodiments, the user may associate an annotation with a specific anatomy in a live image. As the live image displayed changes as an ultrasound probe is moved, the annotation may track the specific anatomy in the display. In some embodiments, the annotation may maintain its association with the specific anatomy by the use of anatomical recognition software included in the imaging system, an example of which may be found in patent application PCT/IB2011/053710, “Automated three dimensional aortic root measurement and modeling.” In some embodiments, an electromagnetic tracking and navigation system such as the PercuNav system may be used to maintain the association of the annotation and specific anatomy. The electromagnetic tracking system may include a field generator which radiates an electromagnetic field permeating the site of imaging and surrounding space. Sensors may be located on the ultrasound probe, the subject being imaged (e.g., patient), and/or other objects (e.g., a biopsy needle). The sensors interact with the electromagnetic field and produce signals used to calculate the position and orientation of the ultrasound probe, subject, and/or objects. The positions calculated by the electromagnetic tracking system may be provided to a graphics processor and/or image processor to coordinate the annotations to the specific anatomy in the image selected by the user.

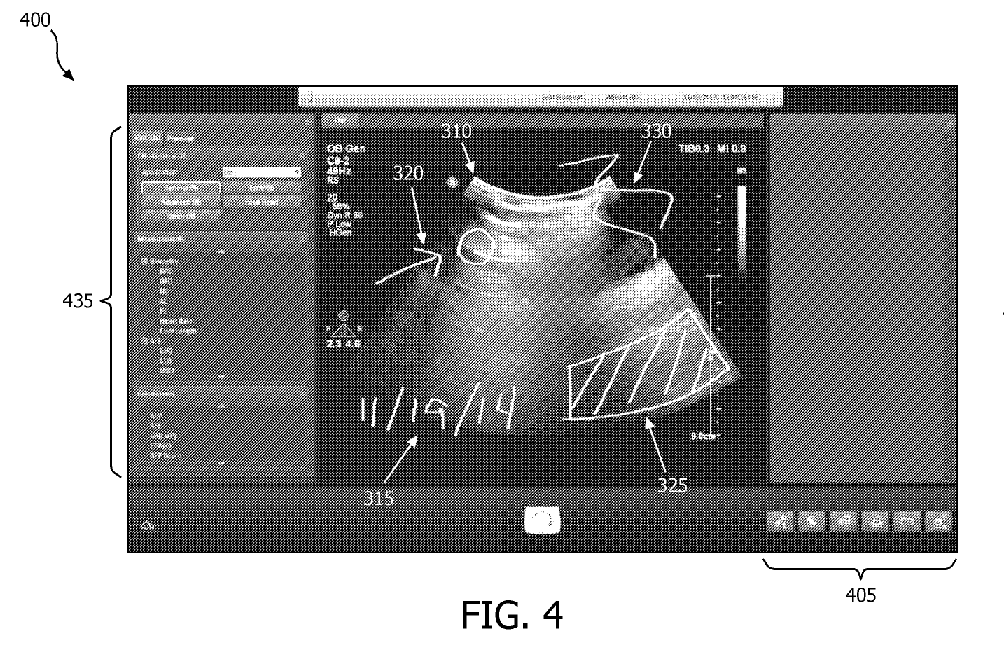

FIG. 4 is an example graphical display 400 of a display of a medical imaging system according to an embodiment of the disclosure. The display 400 may include one or more icons 405 that may be selected by a user to control a medical imaging system. For example, the icons may be used to open help menus, save an image, open previously saved images, and/or other functions. The icons 405 may be selected by a user by using a track ball, mouse, keyboard, and/or other element of a control panel of the medical imaging system. The graphical display 400 may further include an image 310. The image 310 may be the same image displayed on the graphical display 300 of the touch screen shown in FIG. 3. The graphical display 400 may display the same annotations 315, 320, 325, 330 input by a user via the touch screen as described in reference to FIG. 3. The graphical display 400 may include options and menus 435 for controlling the medical imaging system. For example, the user may be able to enter different imaging modes, make measurements, run image analysis software, and/or other applications.

Although not always shown, the graphical displays 300 and/or 400 may also illustrate user selections which may include, for example, icons or menu items which may be selected by the user to, for example, scan, file, print, transfer images (e.g., from one display to another), mute, transcribe, and/or use a headpiece, as desired. Further, one or more menus as is known in the art may be provided for a user's convenience. The displayed images and associated data may be saved at any time during image acquisition or during subsequent analysis. However, a history mode may be activated to gather information indicative of when data may have been added and/or edited so that a user may refer back to original information and/or determine when and/or who made certain changes to information which may be saved in, for example, a generated report. Further, the changes may also be stored for later use.

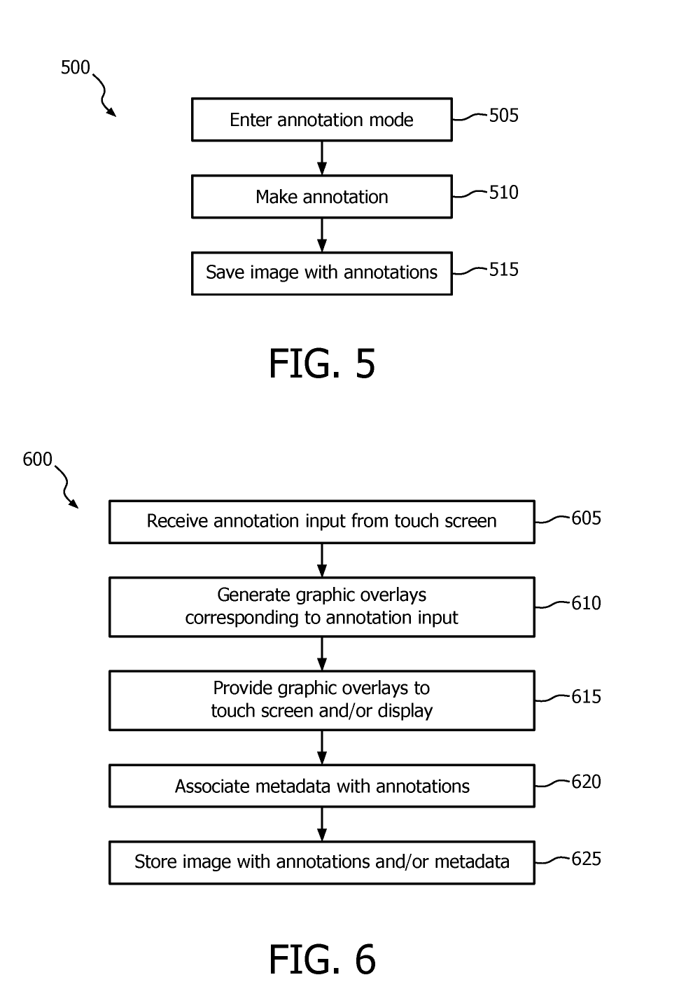

FIG. 5 is a flow chart of a method 500 according to an embodiment of the disclosure. The method 500 may be used to annotate an image acquired by a medical imaging system. In some embodiments, the method 500 may be performed by a user operating a touch screen of the medical imaging system. The user may enter an annotation mode at Step 505. In some embodiments, the user may touch a button on the touch screen to enter the annotation mode. At Step 505, the user may make one or more annotations to the image. In some embodiments, the annotations may be made by the user by interacting with the touch screen. The user may interact with the touch screen by applying a finger, stylus, and/or other input device to the touch screen. At Step 515, the user may save the image with the annotations. The image with annotations may be saved to a memory of the medical imaging system in some embodiments. In some embodiments, the image with annotations may be associated with metadata when saved to memory.

The method 500 may include additional steps in some embodiments. For example, the user may select metadata and/or other data to associate with the annotation made at Step 510. In some embodiments, the annotation may be manually or automatically associated with a specific anatomy of the image prior to or during saving the image with annotations at Step 515.

FIG. 6 is a flow chart of a method 600 according to an embodiment of the disclosure. The method 600 may be used to annotate an image acquired by a medical imaging system. In some embodiments, the method 600 may be performed by a graphics processor of the medical imaging system. At Step 605, the graphics processor may receive input corresponding to annotations for an image acquired by the medical imaging system. The input may be received from a touch screen of the medical imaging system in some embodiments. At Step 610, the graphics processor may generate graphic overlays corresponding to the annotation input received at Step 605. The graphic overlays corresponding to the annotation input may be referred to as annotations. The graphic overlays may be provided to the touch screen and/or a display of the medical imaging system at Step 615. The touch screen and/or display may display the image and annotations received from the graphics processor. Additionally, metadata may be associated with the annotations at Step 620. The metadata may be automatically associated with the annotations by the graphics processor and/or the metadata may be received from an input device such as the touch screen or a control panel. In some embodiments, metadata may be retrieved from a database accessible to the graphics processor. The image with annotations and/or metadata may be stored at Step 625. In some embodiments, the image with annotations and/or metadata may be automatically stored by the graphics processor. In some embodiments, the image with annotations and/or metadata may be stored responsive to a save command from the input device. In some embodiments, the image with annotations and/or metadata may be stored in a memory of the medical imaging system.

In some embodiments, one or more of the steps of method 600 may be omitted. In some embodiments, the image with annotations and/or metadata may not be stored, eliminating Step 625. For example, an ultrasound technician may make annotations to a live image to assist a surgeon during a surgical procedure. The images and annotations made during the procedure may not be relevant for future procedures and/or exams, and the ultrasound technician may choose not to save the images and/or annotations. In some embodiments, Step 620 may be omitted, and no metadata may be associated with the annotations. For example, the medical imaging system may store the image with the annotations as a simple graphic (e.g., a screen shot). The simple graphic may allow limited analysis and/or editing. This may be desirable when little data storage is available and/or only qualitative visual review by a clinician is desired.

Although the present system has been described with reference to an ultrasound imaging system, it is also envisioned that the present system can be extended to other medical imaging systems where one or more images are obtained in a systematic manner. The present system may be used to obtain and/or record image information related to, but not limited to renal, testicular, breast, ovarian, uterine, thyroid, hepatic, lung, muskuloskeletal, splenic, cardiac, arterial and vascular systems, as well as other imaging applications related to ultrasound-guided interventions. Further, the present system may also include one or more programs which may be used with conventional imaging systems so that they may provide features and advantages of the present system.

Further, the present systems, apparatuses, and methods, may also be extended to any small parts imaging where the clear landmarks can be defined and reproduced. Further, the present methods may be embedded in a program code which may be applied to existing imaging systems such as, for example, ultrasonic imaging systems. Suitable ultrasonic imaging systems may include a Philips® ultrasound system which may, for example, support a conventional broadband linear array transducer that may be suitable for small-parts imaging. Further, analysis techniques such as, for example, QLAB™ may be available on-cart with an imaging apparatus or as a post-processing program which may be run outside of an examination room. Further, multiple nodules, anatomical entities such as follicles, or other detectible objects, may be annotated using the present system. Further, the method of the present systems may be applied to volumes acquired using transducers such as, for example, 2D array transducers, which may include, for example, X-matrix™ or mechanical transducers.

Certain additional advantages and features of this invention may be apparent to those skilled in the art upon studying the disclosure, or may be experienced by persons employing the novel system and method of the present invention, chief of which is that a more user friendly image annotation system and method of operation thereof is provided. Another advantage of the present systems and method is that conventional medical image systems can be easily upgraded to incorporate the features and advantages of the present systems, devices, and methods.

Of course, it is to be appreciated that any one of the above embodiments or processes may be combined with one or more other embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A medical imaging system comprising:
a touch screen configured to receive annotation input from a user, wherein the annotation input is associated with an image acquired by the medical imaging system;
a graphics processor configured to receive the annotation input from the touch screen and generate a graphic overlay based, at least in part, on the annotation input, wherein the graphic overlay is associated with the image as an annotation;
a display configured to display the image and the annotation; and
a memory configured to store the image and the annotation.

2. The medical imaging system of claim 1, wherein the graphics processor is further configured to associate metadata with the annotation.

3. The medical imaging system of claim 2, further comprising a database configured to store metadata to associate with the annotation.

4. The medical imaging system of claim 2, wherein the metadata includes at least one of a Digital Imaging and Communications in Medicine (DICOM) meta tag, a specific anatomy, a type of annotation, or a flag.

5. The medical imaging system of claim 1, wherein the image acquired by the medical imaging system is a live image.

6. The medical imaging system of claim 1, wherein the display and the touch screen are configured to display the image and the annotation at the same time.

7. The medical imaging system of claim 1, further comprising an ultrasound probe configured to acquire the image.

8. The medical imaging system of claim 1, further comprising an electromagnetic tracking system configured to calculate positions of a specific anatomy in the image and provide the positions to the graphics processor, wherein the graphics processor is further configured to associate the annotation with the specific anatomy.

9. The medical imaging system of claim 1, wherein the image and the annotation are stored in the memory as separate layers.

10. The medical imaging system of claim 1, wherein the graphics processor comprises a plurality of processors.

11. The medical imaging system of claim 1, wherein the touch screen is configured to control the display.

12. A method of annotating an image, the method comprising:
receiving annotation input from a touch screen, wherein the annotation input is associated with the image;
generating a graphic overlay corresponding to the annotation input, wherein the graphic overlay corresponding to the annotation input is an annotation; and
providing the graphic overlay to the touch screen.

13. The method of claim 12, further comprising associating metadata with the annotation.

14. The method of claim 13, wherein the metadata is automatically associated with the annotations by a graphics processor.

15. The method of claim 13, wherein the metadata to associate with the annotation is received from the touch screen.

16. The method of claim 13, further comprising storing the image, annotation, and metadata in a memory.

17. The method of claim 16, further comprising retrieving the image from the memory based, at least in part, on the metadata.

18. The method of claim 16, further comprising exporting the image, annotation, and metadata from the memory to another device.

19. The method of claim 12, further comprising providing the graphic overlay to a display.

20. The method of claim 12, further comprising entering an annotation mode responsive to an input received from the touch screen.
